(19) United States
(12) Patent Application Publication
(10) Pub. No.: US 2009/0286728 A1
(43) Pub. Date: Nov. 19, 2009

(54) Tooth root formation promoting factors and method for promotion of tooth root formation

(76) Inventors: Masato Ota, Chiba (JP); Nobuyuki Kawashima, Tokyo (JP); Taka Nakahara, Tokyo (JP); Sachiko Iseki, Tokyo (JP)

Correspondence Address: ARTHUR G. SCHAIER;CARMODY & TORRANCE LLP, 50 LEAVENWORTH STREET, P.O. BOX 1110, WATERBURY, CT, 06721, US

(21) Appl. No.: 12/229,786

(22) Filed: Aug. 27, 2008

Related U.S. Application Data
(63) Continuation of application No. PCT/JP2007/053638, filed on Feb. 27, 2007.

(30) Foreign Application Priority Data
Feb. 28, 2006 (JP) 2006-053842

(51) Int. Cl. A61K 38/27; C07K 14/50; A61P 43/00

(52) U.S. Cl. 514/12; 530/399

(57) ABSTRACT

The present invention provides a tooth root formation promoting factor and a method for promotion of tooth root formation, which can promote tooth root formation and which are useful in various aspects of dental therapy. Specifically, the tooth root formation promoting factor contains, as an active ingredient, proteins belonging to the FGF8 subfamily (i.e., fibroblast growth factors); and the method includes administering proteins belonging to the FGF8 subfamily to a mammalian.

### CROSS-REFERENCE TO RELATED APPLICATIONS

This is a continuation of Application No. PCT/JP2007/053638 filed on Feb. 27, 2007.

### BACKGROUND OF THE INVENTION

1. Field of the Invention

The present invention relates to a tooth root formation promoting factor and a method for promotion of tooth root formation, which are useful in various aspects of dental therapy where formation (elongation) of tooth roots is desired.

2. Description of the Related Art

Children, among others, often experience tooth avulsion due to, for example, accidental injuries. Avulsed teeth are recommended to be reduced and fixed as soon as possible. Teeth have thereinside dental pulp tissue rich in nerves and blood vessels and avulsed teeth, in most cases, involve degeneration and devitalization of the dental pulp tissue. In this case, according to a conventional treatment method, the devitalized necrotic tissue is removed and the prepared root canal is filled with calcium hydroxide (see, for example, Int. Dent. J. 2005, October; 55(5): 293-301).

Conventionally in dentistry, apexogenesis refers to a treatment which intends to elongate tooth root and apical closure, whereas apexification refers to a treatment which intends only to close tooth root end using hard tissue. In other words, a treatment which has attained tooth root elongation is called apexogenesis, whereas a treatment which has not attained tooth root elongation is called apexification. The larger the area of a surface where tooth roots face alveolar bone supporting them, the longer and more stable teeth reside in the mouth. Thus, the avulsed teeth, in particular the avulsed root-forming teeth of children, are treated in an attempt to attain apexogenesis; i.e., tooth root elongation.

However, according to the above conventional treatment method, such cases are often encountered that even apexification cannot be attained (needless to say, apexogenesis cannot be attained).

In recent years, there has been positively performed tooth autotransplantation in which an autogenous tooth is transplanted into a portion missing from a tooth. In the tooth autotransplantation, a third molar (a wisdom tooth) or a root-forming third molar is often used. In use of the root-forming third molar, whether or not the transplanted tooth has the same functions as an intrinsic tooth depends on how long the tooth root thereof elongates or how firmly the elongated tooth root is supported by alveolar bone and periodontal ligament. In view of this, it is required to find a new approach for elongating the root of the transplanted tooth or for positively inducing the periodontal tissue to support the tooth root.

One developed technique includes applying FGF2 onto, for example, a tooth root surface for promoting tooth root formation (elongation) (Japanese Patent Application Laid-Open No. 07-17876 and International Publication No. WO2003/082321). Meanwhile, various factors are thought to be involved in tooth root formation in vivo. Thus, demand has arisen for identification of more useful factors in various aspects of dental therapy; e.g., tooth autotransplantation, and treatments for avulsed teeth and periodontal disease.

### BRIEF SUMMARY OF THE INVENTION

An object of the present invention is to solve the problems pertinent in the art and to achieve the following objects. Specifically, an object of the present invention is to provide a tooth root formation promoting factor and a method for promotion of tooth root formation, which are useful in various aspects of dental therapy.

The present inventors carried out extensive research and, as a result, obtained the following findings. Specifically, the present inventors studied on BMP2, FGF2 and FGF18 (known physiologically active substances having bone induction activity) for their tooth root formation promoting activities (tooth root elongation inducing activities) to a root-forming tooth of a mammalian, and have found that FGF18, among others, has remarkably excellent tooth root formation promoting activity. Also, the present inventors, through experiments on functional replacements using an FGF18 knockout mouse, have found that FGF8 belonging to the same subfamily as FGF18 (i.e., the FGF8 subfamily) replaces the function of FGF18. From the above findings, the present inventors have conceived that proteins belonging to the FGF8 subfamily can be effectively used for tooth root elongation, and the present invention has been accomplished.

No reports have been presented on the fact that the proteins belonging to the FGF8 subfamily positively exhibit tooth root formation promoting activity, and this is a quite surprising fact. These proteins can provide a promising approach to dental disease, to which no existing treatment methods are effective and which are expected to attain apexogenesis (a treatment which intends to elongate tooth root and apical closure).

As used herein, “formation” of tooth roots refers not only to formation of new tooth roots but also to “elongation” of existing tooth roots, and “elongation” of tooth roots refers not only to elongation of existing tooth roots but also to “formation” of new tooth roots. Thus, “formation” and “elongation” are herein equivalent to each other, and this shall apply to expressions equivalent to any of these.

The present invention is based on the above findings obtained by the present inventors, and provides the followings.

<1> A promoting factor for formation of a tooth root and a periodontal tissue supporting the tooth root including, a protein belonging to the FGF8 subfamily as an active ingredient.
<2> The promoting factor according to claim 1, wherein the protein belonging to the FGF8 subfamily is FGF18.
<3> The promoting factor according to claim 1, wherein the protein belonging to the FGF8 subfamily is FGF8.
<4> The promoting factor according to claim 1, wherein the protein belonging to the FGF8 subfamily is FGF17.
<5> A method for promoting formation of a tooth root and a periodontal tissue supporting the tooth root including, administering a protein belonging to the FGF8 subfamily to a mammalian.
<6> The method according to claim 5, wherein the protein belonging to the FGF8 subfamily is FGF18.
<7> The method according to claim 5, wherein the protein belonging to the FGF8 subfamily is FGF8.
<8> The method according to claim 5, wherein the protein belonging to the FGF8 subfamily is FGF17.

The present invention can solve the aforementioned problems, and provides a tooth root formation promoting factor and a method for promotion of tooth root formation, which are useful in various aspects of dental therapy.

### BRIEF DESCRIPTION OF THE SEVERAL VIEWS OF THE DRAWINGS

FIG. 1 shows photographs in relation to transplant culturing which includes subrenally transplanting extirpated teeth having embedded FGF18-impregnated beads.

FIG. 2 is a graph showing the length of the formed (elongated) tooth root, after transplant culturing, of each extirpated tooth with a physiologically active substance (BMP2, FGF2 or FGF18)-impregnated bead being embedded.

FIG. 3 is a photograph of an extirpated tooth having an embedded FGF18-impregnated bead, wherein the photograph is obtained using soft X-ray photography after transplant culturing.

FIG. 4 shows images of an extirpated tooth having an embedded FGF18-impregnated bead after transplant culturing, wherein the images are obtained after histological staining and the characters B, C, D, PL, CB and OB denote bone, cementum, dentine, periodontal ligament, cementoblasts and osteoblasts, respectively.

FIG. 5 is photographs showing GFP-positive bone formation in a GFP-mouse-derived tooth having an embedded FGF18-impregnated bead after transplant culturing.

FIG. 6 is photographs showing, after transplant culturing, extirpated teeth with physiologically active substance (FGF2 or FGF18)-impregnated beads being embedded, periodontal bone and neovascularization.

FIG. 7 is an electrophoretic profile in relation to endogenous FGF18 expression in dental pulps (including tooth roots) of incisors of mice and rats which continue to form tooth roots.

FIG. 8 is photographs showing, after transplant culturing, extirpated teeth having embedded physiologically active substance (FGF8)-impregnated beads, periodontal bone and neovascularization, wherein white lines indicate profiles of elongated tooth roots.

### DETAILED DESCRIPTION OF THE INVENTION

### Tooth Root Formation Promoting Factors

A tooth root formation promoting factor of the present invention contains proteins belonging to the FGF8 subfamily (FGF8 subfamily proteins) as an active ingredient and, if necessary, appropriately contains other ingredients.

### <Proteins Belonging to the FGF8 Subfamily>

Fibroblast growth factors (FGFs) are classified into seven subfamilies according to structural similarity. FGF18, which belongs to the FGF8 subfamily including FGF8 and FGF17, is known as a physiologically active substance having bone formation stimulating activity. However, no reports have been presented concerning its effects on tooth roots.

The present inventors have first found that FGF18 has excellent tooth root formation promoting activity. Also, through experiments on functional replacements using FGF18 knockout mice, it has been found that FGF8 belonging to the same subfamily as FGF18 (i.e., the FGF8 subfamily) replaces the functions of FGF18. Thus, the FGF8 subfamily proteins may be used as an active ingredient in the present tooth root formation promoting factors.

The FGF8 subfamily proteins are not particularly limited and appropriately selected depending on the purpose. Examples include recombinant human FGF8 subfamily proteins, recombinant mouse FGF8 subfamily proteins, and those derived from other mammalians. When used in a human for treatment, the FGF8 subfamily proteins are particularly preferably derived from a human. Notably, in the sequence listing given below, SEQ ID Nos: 1 and 2 correspond to human FGF18; SEQ ID Nos: 3 and 4 correspond to human FGF8; and SEQ ID Nos: 5 and 6 correspond to human FGF17.

The FGF8 subfamily proteins may be a purified extract derived from, for example, living organisms; or may be produced from microorganisms or culture cells with a known recombinant protein production technique. Also, in addition to the purified extract, an unpurified cell extract, etc. may be used as the FGF8 subfamily proteins.

Furthermore, the FGF8 subfamily proteins may be a commercially available one, and examples include those of, for example, PeproTech Co. and Sigma Co.

The FGF8 subfamily proteins are not particularly limited, so long as they have the intrinsic physiological activities thereof and have tooth root formation promoting activity. For example, there may be used polypeptides having an amino acid sequence substantially identical to those of naturally occurring FGF8 subfamily proteins. Notably, the polypeptides are those having the physiological activities of the naturally occurring FGF8 subfamily proteins and being identical thereto in amino acid sequence, except that several amino acids are substituted. Also, fragments of the polypeptides may be used as the FGF8 subfamily proteins, so long as they have the physiological activities of the naturally occurring FGF8 subfamily proteins and have tooth root formation promoting activity. Furthermore, the polypeptides or fragments may be modified at their N- and/or C-terminuses, so long as they have the physiological activities of the naturally occurring FGF8 subfamily proteins and have tooth root formation promoting activity.

The FGF8 subfamily proteins may be used alone or in combination.

The FGF8 subfamily protein content of the tooth root formation promoting factors is not particularly limited, and can be appropriately determined in consideration of the selected dosage form of the tooth root formation promoting factors. The content is preferably 0.025% by mass to 3.0% by mass, more preferably 0.1% by mass to 1.0% by mass, particularly preferably 0.25% by mass to 0.5% by mass.

### <Other Ingredients>

The tooth root formation promoting factors may be the FGF8 subfamily protein itself, or a product containing the FGF8 subfamily protein and other ingredients.

The other ingredients are not particularly limited and can be appropriately selected depending on the purpose so as not to impede the effects of the present invention. Examples thereof include pharmaceutically acceptable carriers. The pharmaceutically acceptable carriers are not particularly limited and can be appropriately selected in consideration of, for example, the selected dosage form of the tooth root formation promoting factors. Examples thereof include solvents, isotonicity agents, emulsifiers, suspending agents, stabilizers and fillers, which are generally used in drugs for use in dental therapies.

In particular, heparin-containing absorbable beads or gel is preferred as the pharmaceutically acceptable carrier, since the FGF8 subfamily protein has an affinity to heparin. The heparin-containing absorbable beads or gel may be commercially available from, for example, Sigma Co.

The other ingredients content is not particularly limited and can be appropriately determined depending on the purpose so as not to impede the effects of the present invention. For example, the content may be appropriately determined so as to attain the desired FGF8 subfamily protein (active ingredient) content of the tooth root formation promoting factors.

### <Dosage Form>

The dosage form is not particularly limited and can be appropriately selected depending on the purpose from known dosage forms. For example, the FGF8 subfamily protein and the carrier appropriately selected from the above carriers are formed into an external preparation (e.g., liquid, emulsion, or a gelling agent), an impregnating agent, an adhesive preparation, injection or powder. The method for forming the protein and carrier into any of the above dosage forms is not particularly limited and can be appropriately selected depending on the purpose from known forming methods.

In particular, preferred are heparin-containing absorbable beads or gel which is impregnated with the FGF8 subfamily protein. The FGF8 subfamily protein-impregnated heparin-containing absorbable beads or gel can provide tooth root formation promoting factors having controlled-release properties. This is advantageous in that the tooth root formation promoting factor is not lost from an application site in a short time due to outflow, etc., and that the FGF8 subfamily protein serving as an active ingredient can continue to act on the application site for a relatively long period of time (preferably for two to three days).

The method for forming the FGF8 subfamily protein-impregnated heparin-containing absorbable beads is not particularly limited. For example, they can be formed as follows: the FGF8 subfamily protein is diluted with phosphate buffered saline (PBS); and the resultant solution was added to commercially available heparin-containing absorbable beads; and the resultant beads are incubated (shown in Examples given below).

The method for forming the FGF8 subfamily protein-impregnated heparin-containing absorbable gel is not particularly limited. For example, it can be formed as follows: a heparin solution is added to collagen gel formed predominantly of type I collagen; and heparin is electrostatically bonded to the type I collagen in PBS, followed by incubation (see, for example, Biochim. Biophys. Acta. (1986) 882: 1-5).

The tooth root formation promoting factor can be provided with desired controlled-release properties by appropriately determining the amount of a solution of the FGF8 subfamily protein and the employed carrier. The controlled-release tooth root formation promoting factor enables the FGF8 subfamily protein, serving as an active ingredient, to act on an application site for a period of time that is determined depending on the symptoms, etc. of a patient in need thereof.

### (Method for Promotion of Tooth Root Formation)

A method for promotion of tooth root formation of the present invention includes administering the FGF8 subfamily proteins to a mammalian and, if necessary, includes other steps.

In the method for promotion of tooth root formation, the form of the FGF8 subfamily protein administered to a mammalian is not particularly limited. The FGF8 subfamily protein may be administered as is, or may be administered in the form of FGF8 subfamily protein-impregnated heparin-containing absorbable beads or gel (i.e., a preferred dosage form of the tooth root formation promoting factors). Use of the FGF8 subfamily protein-impregnated heparin-containing absorbable beads or gel is particularly preferred, since the FGF8 subfamily protein can act on an administration site thereof in a mammalian for a relatively long period of time.

### <Mammalian>

The mammalian to which the tooth root formation promoting factor (FGF8 subfamily protein) is administered is not particularly limited and can be appropriately selected depending on the purpose. Examples thereof include humans, pet animals, livestock animals and experimental animals (e.g., monkeys, bovines, pigs, mice, rats, dogs and cats).

### <Administration>

The site to which the tooth root formation promoting factor (FGF8 subfamily protein) is administered is not particularly limited, so long as formation of a tooth root of interest can be promoted. For example, the promoting factor (protein) can be applied to a dental pulp, a tooth root surface, an alveolar bone surface, or sites in the vicinity thereof. The administration method of the tooth root formation promoting factor (FGF8 subfamily protein) is not particularly limited and can be appropriately selected from known administration methods in consideration of, for example, the dosage form of the tooth root formation promoting factor. For example, the promoting factor (protein) may be embedded in or injected to a dental pulp; or may be coated on a tooth root surface or an alveolar bone surface.

The administration dose of the tooth root formation promoting factor (FGF8 subfamily protein) is not particularly limited and can be appropriately determined in consideration of various factors such as the symptoms, constitution, age and body weight of a target subject, and his/her concomitant medication. For example, the promoting factor (protein) is preferably administered to a human adult at a dose of 0.01 mg to 1.0 mg, more preferably 0.05 mg to 0.5 mg, particularly preferably 0.1 mg to 0.3 mg, wherein each administration dose is a dose at which the promoting factor (protein) is singly administered to one site, and is reduced to the amount of the FGF8 subfamily protein (active ingredient). Also, the dose at which the promoting factor (protein) is administered to the other mammalians is not particularly limited and can be appropriately determined in consideration of the above various factors. In general, the administration dose for the other mammalians may be determined by reducing the administration dose for the human adult on the basis of the body weight. Particularly preferably, the promoting factor (protein) is administered to a target subject at a dose of 2.5 μg/kg. Furthermore, the number at which the promoting factor (protein) is administered per day is not particularly limited and can be appropriately determined in consideration of the above various factors.

The present tooth root formation promoting factor containing, as an active ingredient, the FGF8 subfamily proteins have excellent tooth root formation promoting activity. Thus, when administered to a mammalian, the present promoting factor can promote formation of tooth roots thereof. Also, the present tooth root formation promoting factor can be provided with desired controlled-release properties by appropriately determining the employed carrier and the amount of the FGF8 subfamily proteins serving as an active ingredient, and the controlled-release tooth root formation promoting factor can act on an application site for a desired period of time. Furthermore, the present tooth root formation promoting factor has excellent safety, since it employs safe FGF8 subfamily proteins as an active ingredient.

Thus, the present tooth root formation promoting factor and the present method for promotion of tooth root formation, which employ the FGF8 subfamily protein, can be expected to be clinically applied to various aspects of dental therapy where promotion of tooth root formation is desired. Conceivably, the present promoting factor and method are particularly advantageously used as the followings (1) to (7):

(1) use thereof in pediatric dentistry can induce tooth root elongation in root-forming teeth, which have suffered from disorders caused by trauma or infection in the process of tooth root formation and have been prevented from elongating;
(2) use thereof in orthodontic dentistry can induce regeneration of tooth roots resorbed due to orthodontic force accompanying orthodontic therapy;
(3) use thereof in general dentistry can induce regeneration of tooth roots resorbed caused by lesions in root apices (e.g., radicular cyst);
(4) use thereof in general dentistry can induce regeneration of tooth root-periodontal tissue after intentional reimplantation for the treatment of, for example, root fracture;
(5) use thereof in oral surgery can promote formation of tooth root-periodontal tissue of autotransplanted supernumerary teeth (third molars (i.e., wisdom teeth));
(6) use thereof in regenerative medicine can be expected to induce regeneration of tooth root-periodontal tissue after autotransplantation of juvenile teeth which have been stored in a frozen state; and
(7) use thereof in general dentistry can be expected to induce regeneration of cementum, periodontal ligament and alveolar bone resorbed due to, for example, periodontal disease.

As shown in Examples given below, the present tooth root formation promoting factors and the present method for promotion of tooth root formation exhibit not only tooth root formation promoting activity but also an activity of promoting formation of periodontal tissues supporting tooth roots; e.g., periodontal ligament and alveolar bone. Thus, the present promoting factors and method are expected to be clinically applied to a wider variety of aspects of dental therapy.

Also, the present tooth root formation promoting factors and the present method for promotion of tooth root formation can be applied not only to apexogenesis therapy intending to elongate tooth root and closure root apex but also to apexification therapy intending only to close tooth root apex using hard tissue. Thus, the present promoting factors and method are expected to be used in a wider variety of clinical applications.

### EXAMPLES

The present invention will next be described by way of examples, which should not be construed as limiting the present invention thereto.

### Example 1

### FGF18

### Preparation of FGF18-Impregnated Heparin-Containing Absorbable Beads

FGF18-impregnated heparin-containing absorbable beads (a preferred embodiment of a tooth root formation promoting factor of the present invention) were prepared as follows.

FGF18 (product of PeproTech Co.) was diluted with Dulbecco's modified phosphate buffered saline (D-PBS) to a concentration of 250 μg/mL. Heparin acrylic beads (product of Sigma Co.), which had been provided as heparin-containing absorbable beads, were washed with phosphate buffered saline (PBS). The obtained FGF18-diluted solution was added to the thus-washed heparin acrylic beads, followed by incubation at 37° C. for 45 minutes, whereby the heparin acrylic beads were impregnated with FGF18. The obtained FGF18-impregnated heparin acrylic beads were stored at 4° C. before use. In use, after washed with D-PBS so as to remove excessive FGF18, the beads were set in dental pulps as described below.

### <Embedment in Dental Pulp>

Five-day-old C57BL/6 mice, at a stage when their tooth roots were about to be formed, were etherized, followed by decapitation. After removal of the mandibles, the mandibular first molars were extirpated therefrom together with the dental follicles. Thereafter, the FGF18-impregnated heparin acrylic beads were set in the dental pulps of some extirpated teeth (mandibular first molars) so that one bead was set in the dental pulp of one extirpated tooth.

Meanwhile, D-PBS alone was injected into the dental pulps of other extirpated teeth (mandibular first molars), which were used as a negative control.

### <Transplant Culturing>

The FGF18-impregnated heparin acrylic bead-embedded teeth and the D-PBS-injected teeth (serving as a negative control) were subrenally transplanted into host mice, followed by culturing. Three weeks after, each of the host mice was killed with cervical dislocation, and the kidney was removed to recover the cultured teeth (transplanted teeth). Notably, the host mice were 10- to 15-week-old C57BL/6 male mice, since nude mice were not particularly required in this transplant culturing. This is because, among organs of an adult mouse, the kidney and the camera anterior bulbi are known to exhibit considerably lower immune response.

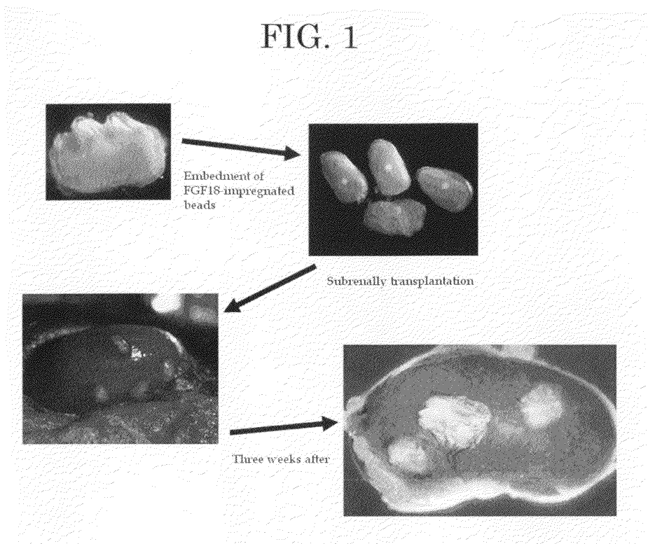

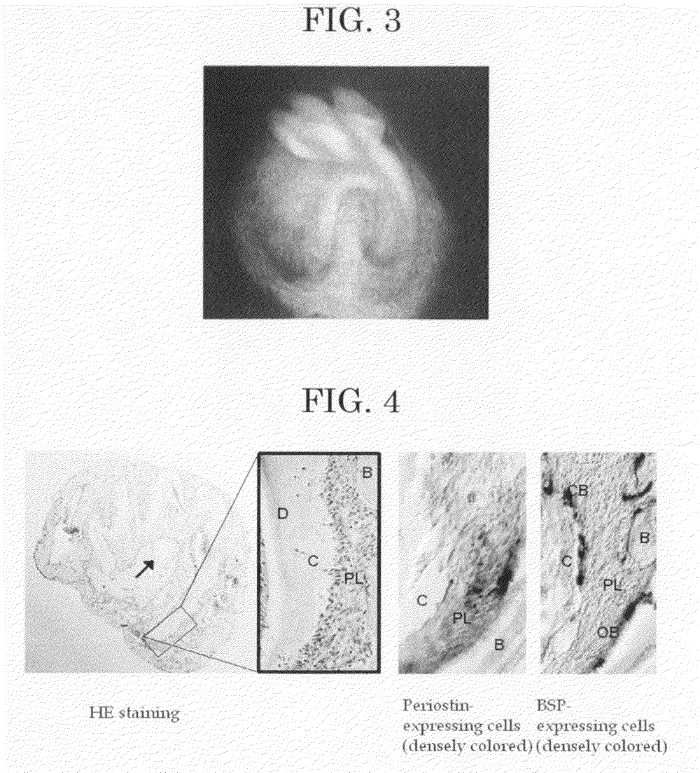

The recovered transplanted teeth were observed under a stereomicroscope for their tooth root formations (elongations). FIG. 1 shows photographs in relation to the transplant culturing. FIGS. 3 to 6 show photographs of the transplanted teeth, periodontal bone and neovascularization.

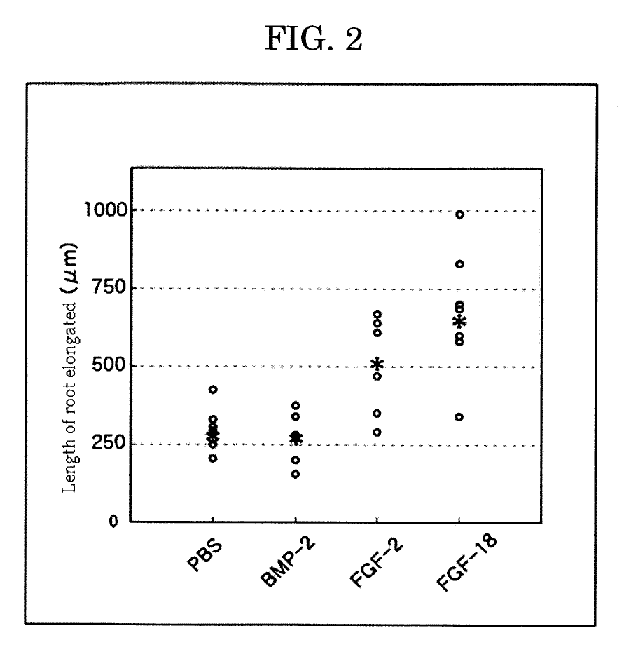

The formed (elongated) tooth roots of the recovered transplanted teeth were photographed with a stereofluorescence microscope system VBG-25 (product of KEYENCE CORPORATION) to obtain digital images thereof. The length (μm) of the formed (elongated) tooth roots was measured, while observing the obtained digital images, using an automatic measuring software attached to the system. The results are shown in FIG. 2.

### Comparative Example 1

### BMP2

The procedure of Example 1 was repeated, except that BMP2 (product of R&D Co.) was used instead of FGF18, the concentration of the diluted solution was changed to 500 ng/mL, and Affi-Gel agarose beads (product of Bio-Rad Laboratories, Inc.) were used instead of the heparin acrylic beads, to thereby prepare BMP2-impregnated agarose beads of Comparative Example 1.

Subsequently, similar to Example 1, the BMP2-impregnated agarose beads of Comparative Example 1 were embedded in the dental pulps, followed by transplant culturing. Also, the teeth obtained after transplant culturing were measured for the length of the formed (elongated) tooth roots. The results are shown in FIG. 2.

### Comparative Example 2

### FGF2

The procedure of Example 1 was repeated, except that FGF2 (product of PeproTech Co.) was used instead of FGF18, to thereby prepare FGF2-impregnated heparin acrylic beads of Comparative Example 2.

Subsequently, similar to Example 1, the FGF2-impregnated heparin acrylic beads of Comparative Example 2 were embedded in the dental pulps, followed by transplant culturing. Also, the teeth obtained after transplant culturing were measured for the length of the formed (elongated) tooth roots. The results are shown in FIG. 2. In addition, the teeth, periodontal bone and neovascularization were observed. The results are shown in FIG. 6.

### Results

### Example 1 and Comparative Examples 1 and 2

From the data shown in FIG. 2, the teeth of the negative control group using D-PBS alone (PBS in FIG. 2) and the experimental group using BMP2 (Comparative Example 1; BMP-2 in FIG. 2) were found to somewhat exhibit tooth root elongation. The teeth of the experimental group using FGF2 (Comparative Example 2; FGF-2 in FIG. 2) were found to exhibit tooth root elongation somewhat significantly greater than those of the negative control group and the BMP2-experimental group, but the length of the elongated tooth roots was found to be less than that of the experimental group using FGF18 (Example 1; FGF-18 in FIG. 2). Thus, the teeth of the FGF18-experimental group were found to exhibit tooth root elongation remarkably greater than those of any other experimental groups.

Notably, in FIG. 2, the symbol “o” corresponds to measurements for transplanted tooth samples of each group in this experiment, and the symbol “*” corresponds to mean values of the measurements.

The transplanted tooth samples of Example 1, which have been embedded with FGF18-impregnated heparin acrylic beads and exhibited promoted tooth root elongation, were found to exhibit bone formation around the formed tooth roots. The formed bone was subjected to soft X-ray photography to determine whether or not the tooth roots of the transplanted teeth elongated thereinside. From the obtained photographs, the tooth roots were found to elongate inside the formed bone (FIG. 3). In addition, through histological observation with HE staining, periodontal ligament-like fibroblasts were found between the formed tooth roots and the periodontal bone (a left-hand photograph of FIG. 4). In the left-hand photograph of FIG. 4, the embedded FGF18-impregnated heparin acrylic bead is indicated by an arrow. Furthermore, through in situ hybridization, these periodontal ligament-like fibroblasts were found to express the periostin gene, which is a marker gene of the periodontal ligament (a middle photograph of FIG. 4), and also, bone siaroprotein (BSP)-positive cells were found to be present on the surface of the bone around the tooth roots (a right-hand photograph of FIG. 4). In the middle and right-hand photographs of FIG. 4, densely colored are areas where cells expressing the periostin gene and producing the bone siaroprotein are present.

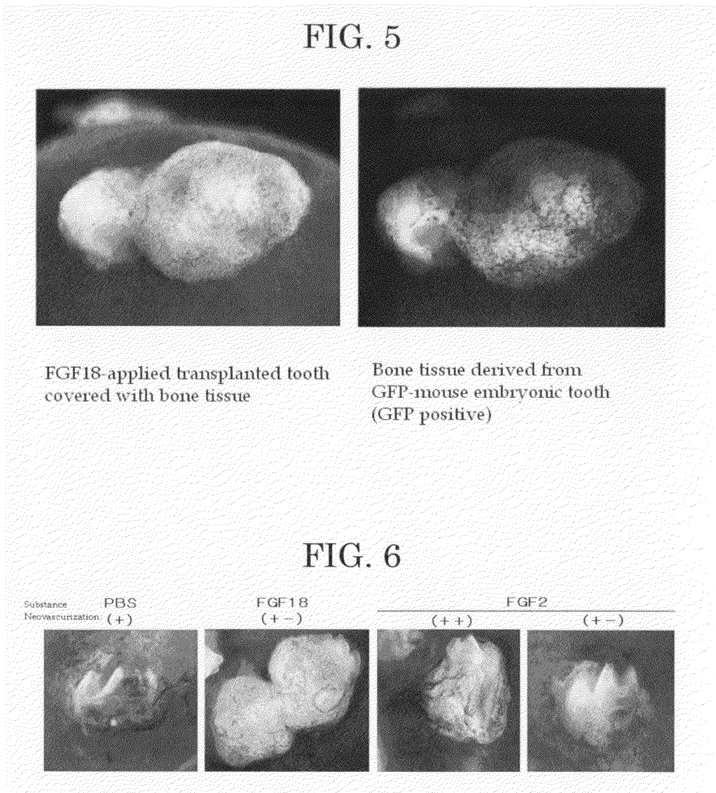

Separately, when FGF18 was applied together with an embryonic tooth of a green fluorescent protein (GFP)-mouse serving as a donor tooth, the bone formed around a transplanted tooth (a left-hand photograph of FIG. 5) was found to be derived from tissue thereof with a stereofluorescence microscope MZFLIII (product of Leica MICROSYSTEMS) (a right-hand photograph of FIG. 5).

From photographs shown in FIG. 6, the teeth of the negative control group using D-PBS alone (in FIG. 6, a photograph specified by PBS and neovascularization (+−)) and the FGF2-experimental group that did not considerably exhibit neovascularization (Comparative Example 2; in FIG. 6, a photograph specified by FGF2 and neovascularization (+−)) were found to somewhat exhibit tooth root elongation. The teeth of the FGF2-experimental group that considerably exhibited neovascularization (Comparative Example 2; in FIG. 6, a photograph specified by FGF2 and neovascularization (++)) were found to be significantly greater in tooth root elongation and periodontal bone formation than those of the negative control group and the other FGF2-experimental group, but the degrees of the elongation and formation were found to be less than those of the FGF18-experimental group (Example 1; in FIG. 6, a photograph specified by FGF18 and neovascularization (+−)). Meanwhile, although the teeth of the FGF18-experimental group did not considerably exhibit neovascularization, they were considerably promoted in tooth root elongation and periodontal bone formation, as compared with the other experimental groups.

The above discussion indicates that the present tooth root formation promoting factor containing FGF18 as an active ingredient has excellent tooth root formation (elongation) activity and also, has an activity of promoting formation of periodontal tissues supporting the formed tooth root; e.g., periodontal ligament and alveolar bone.

### Referential Example 1

### Expression of Endogenous FGF18 Gene

In Referential Example 1, a mammalian, at a stage when tooth root formation was initiated, was examined as to whether or not endogenous FGF18 expressed in the tooth roots (including dental pulps).

Incisors were extracted from ten-week-old mice, which are known to continue to form tooth roots. Under a stereomicroscope, root apex tissue and dental pulps of root-crown were separated from each incisor. The thus-obtained root apex tissue samples and dental pulp samples were stored at 4° C. in the presence of RNAlater (product of QIAGEN) exhibiting RNase inhibitory activity.

Total RNA was extracted from each of the thus-obtained root-crown samples with QuickGene (product of Fuji Photo Film Co. Ltd.). Specifically, the root-crown sample (including hard tissue) was thoroughly pulverized with a disposable plastic pestle in the presence of a lysis buffer, followed by centrifugation. Thereafter, the supernatant was mixed with a predetermined amount of ethanol, and then the resultant mixture was applied to the QuickGene column. The extracted RNA was analyzed with a spectrophotometer for the concentration and 260/280 ratio. Subsequently, cDNA of the root-crown sample was prepared using RNA samples (300 ng) exhibiting a 260/280 ratio of 1.8 or higher. In this cDNA preparation, reverse transcription was carried out using SuperScriptII (product of Invitrogen Corp.). The FGF18 expression level of the sample was determined by quantitative polymerase chain reaction (qPCR). Specifically, the FGF18-specific primers were fabricated using design software on the Internet (see http://frodo.wi.mit.edu/cgi-bin/primer3/primer3_www.cgi); the quantitative PCR was carried out using Platinum SYBR Green qPCR SuperMix-UDG (product of Invitrogen Corp.) and DNA engine Opticon (product of Bio-Rad Laboratories, Inc.); and the FGF18 expression level was evaluated by comparing with that of β-actin serving as an internal standard substance.

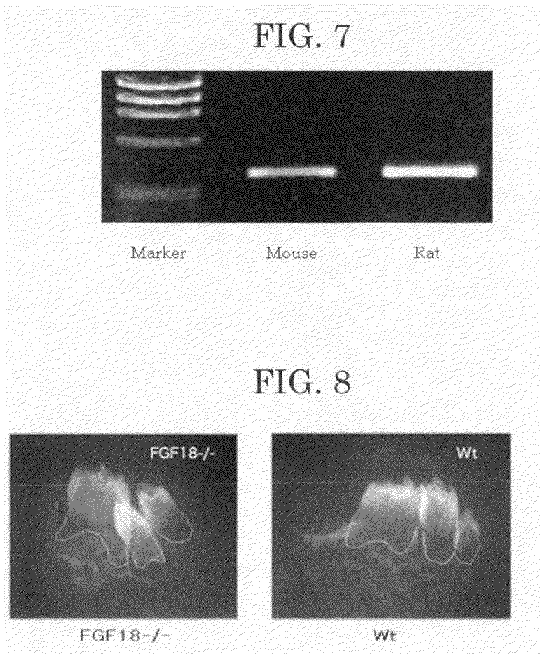

Similarly, six-week-old rats, at a stage when tooth root formation was initiated, were treated, to thereby evaluate endogenous FGF18 expression levels in dental pulps of root-crown. The results are shown in FIG. 7.

From the data obtained from the quantitative PCR, endogenous FGF18-gene expression was observed in tooth roots (including dental pulps) of mice and rats forming tooth roots (FIG. 7). Notably, the fact that FGF18 specifically expresses in elongating tooth roots (including dental pulps) of a mammalian has not conventionally known, and this fact has newly been found out by the present inventors.

### Example 2

### FGF8

The procedure of Example 1 was repeated, except that FGF8 (product of PeproTech Co.) was used instead of FGF18, to thereby prepare FGF8-impregnated heparin acrylic beads of Example 2 (a preferred embodiment of the present tooth root formation promoting factor).

Similar to Example 1, the FGF8-impregnated heparin acrylic beads of Example 2 were embedded in the dental pulps of the embryonic teeth extirpated from wild-type mice, followed by transplant culturing. Also, the teeth obtained after transplant culturing and the periodontal bone were observed with soft X-ray photography. Separately, the above procedure was repeated, except that the embryonic teeth extirpated from the wild-type mice were substituted with embryonic teeth extirpated from FGF18-gene knockout embryonic mice (embryonic age: 18.5 days). The results are shown in FIG. 8.

### Results

### Example 2

The teeth of the experimental group in which FGF8 was applied to the embryonic teeth extirpated from the wild-type mice were found to exhibit tooth root elongation (Example 2; in FIG. 8, a photograph specified by Wt), indicating that FGF8 has tooth root elongation promotion activity similar to FGF18. Also, the teeth of the experimental group in which FGF8 was applied to the embryonic teeth extirpated from the FGF18-gene knockout mice were found to exhibit tooth root elongation (in FIG. 8, a photograph specified by FGF18−/−), indicating that FGF8 has an activity of replacing physiological functions of FGF18.

The data obtained from Example 1 and Comparative Examples 1 and 2 indicate that the present tooth root formation promoting factor containing FGF18 as an active ingredient very effectively promotes formation of tooth roots and periodontal tissues supporting them; e.g., periodontal ligament and alveolar bone.

Also, from the data obtained from Referential Example 1, FGF18 is first elucidated to be a important factor involved in tooth root formation of a mammalian at a stage when tooth root formation is initiated, indicating that the present tooth root formation promoting factor containing FGF18 as an active ingredient has excellent tooth root formation promoting activity.

Further, from the data obtained from Example 2, FGF8 has the same tooth root formation promoting activity as FGF18, suggesting that effectiveness of FGF8 family proteins (FGF8, FGF17 and FGF18) in promoting formation of tooth roots and periodontal tissues supporting them; e.g., periodontal ligament and alveolar bone.

The present tooth root formation promoting factors and the present method for promotion of tooth root formation can be expected to be clinically applied to various aspects of dental therapy where promotion of tooth root formation is desired. Conceivably, the present promoting factors and method are particularly advantageously used as the followings (1) to (7):

(1) use thereof in pediatric dentistry can induce tooth root elongation in root-forming teeth, which have suffered from disorders caused by trauma or infection in the process of tooth root formation and being prevented from elongating;
(2) use thereof in orthodontic dentistry can induce regeneration of tooth roots resorbed due to orthodontic force accompanying orthodontic therapy;
(3) use thereof in general dentistry can induce regeneration of tooth roots resorbed caused by lesions in root apices (e.g., radicular cyst);
(4) use thereof in general dentistry can induce regeneration of tooth root-periodontal tissue after intentional reimplantation for the treatment of root fracture, etc.;
(5) use thereof in oral surgery can promote formation of tooth root-periodontal tissue of autotransplanted third molars (i.e., wisdom teeth);
(6) use thereof in regenerative medicine can be expected to induce regeneration of tooth root-periodontal tissue after autotransplantation of juvenile teeth which have been stored in a frozen state; and
(7) use thereof in general dentistry can be expected to induce regeneration of cementum, periodontal ligament and alveolar bone resorbed due to, for example, periodontal disease.

What is claimed is:

1. A promoting factor for formation of a tooth root and a periodontal tissue supporting the tooth root comprising, a protein belonging to the FGF8 subfamily as an active ingredient.

2. The promoting factor according to claim 1, wherein the protein belonging to the FGF8 subfamily is FGF18.

3. The promoting factor according to claim 1, wherein the protein belonging to the FGF8 subfamily is FGF8.

4. The promoting factor according to claim 1, wherein the protein belonging to the FGF8 subfamily is FGF17.

5. A method for promoting formation of a tooth root and a periodontal tissue supporting the tooth root comprising, administering a protein belonging to the FGF8 subfamily to a mammalian.

6. The method according to claim 5, wherein the protein belonging to the FGF8 subfamily is FGF18.

7. The method according to claim 5, wherein the protein belonging to the FGF8 subfamily is FGF8.

8. The method according to claim 5, wherein the protein belonging to the FGF8 subfamily is FGF17.


SEQUENCE LISTING (6 sequences)

SEQ ID NO: 1 - DNA, 1546 bp, human
  CDS (538)..(1161)
cacggccggagagacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaaaccaaagtcctttttcccaaaggttctgaaaggaaaaaaaaaaaaaaacaaaaaaaaaaaaaaaa

SEQ ID NO: 2 - protein, 207 aa, human
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 3 - DNA, 9714 bp, human
  CDS (2091)..(2122)
acttgtgtccacctgacttgtctccgcctgatggacccacctccccatgggctttagaggagaccagtgcctgccaagcctgtggcagatacaagtgctcactaactggtggtgttcctggtgataatctgttgggaggaggtgaaggcaggtcttcctggggaagttgcaaataggctgaagtctacaggctcagccacttcccccatcattcattcattcattcattcaacgaactcttaattgcagaacagctctgcccagccttgtgccacgagctgtgatttctgagctcataagacatggtccttgtgggcaaggtctgtgtgggcaagagcccactgaacagactcctggaggaggatggcaagtgtgctctggaagtctacttctctgaggagggataagatcttggaacgcaccctctaactgttttcccatctctccattgctcagtgtttgcatttatggctgggagttggaaagatggggcacagggaaaggtgggacacagagcagggagggaggcagggcctggctgtgctgctggactagggctgccgctaattttggcctaatggcttcctgggaccacaccaaaccctccagccctacccccaggggaatggagggtcactggggaaggggaggttgtaagtgaagacagcagtctggtagggcatcacctgctcctcacaaggcacctgtcccagagcatctctgtccacccaggctgctcccacagaagcagcatgggggctcagggtttccagaaacctcaggggggaagccacaaggtggggggctaggaaaggcttagacaagtgcctaccttctcctatgccccaagcccagctgaccagcccctgcctagaatacagtaggtgtgtaaatacctgtgaaatgaaaaacgagtggggctttgagggggaaagggctgggtggcctggatggaagtcccttcccccagtaaaagctactgagagtttttttgcccctgctgcctgctgaggtacggggacagtgggtcctgccagggacctccggactctctcttccacacaggtggctcggggtgctcctcaagcttcaggctagtgtgtgtgtgtgtgtgtgtgtgtgtgtgtgtgtgtgtggtgaggggcacaggggaaggggtgatagtgataatgggatggggggagggggaggcatttctggctcccagggcctcctcgggagagtgaaggagactggagagaccgacaggacggggcacgcggccgaaacaggggagcggggtccgggtggcaccggcacgcactgcgcgagggccgggtgcgcagccattcgctgcatggttagcggccgctcccaggggcccggcgggacacacagtaggtggctcggagaccactgcgtagccggcggcggaagcggctgggcccggaggcggagacccagacccgccagactcgggcgctccaggcggtcggggaagcccaggctcggggtctcccggcccgcagagtttgcagccctaggcgcccccatccccgcgcgtccgggaccggggagcgcgcgaggggacaggccggggatagggggaggcgcccagaccggccgcccccgcccctcccgcgggtccccagacccctcccctagcccagaccctcaatcccgaacccgtcggccgcccctccccggccgccccgcccccgccccgggctgcatccaggccccagttcccagccgggggctgcgggctcccgccgcccgccccgcgctcccccctcgcgcccggtgcggcgccccccgcccccacccctccccgccctcccacctccccacccaccccgccagcccgtgaccctcccgcccgctccggctcccgagcagccagcggccaccggctccggcagcggcgcacagcgattcggtgcggcgcggcgagcacgacgttccacgggacccgcggagccgcgtcgtgatcgccgccggcctcccgcacccgcaccctctccgctcgcgccctgctcagcgcgtcctcccgcggcggcccgcgggacggcgtgacccgccgggctctcggtgccccggggccgcgcgccatgggcagcccccgctccgcgctgagctgcctgtgagtaccgcgccgccctgccccgccacccgccccccgcgcccctcgcctcacccgcgcctctctctcccgcccgcttttgtctcccacaggctgttgcacttgctggtcctctgcctccaagcccaggtgaggaggggtgcgcggaggcgggggccgggcgcgccggtgtgagacccgggtgggcagcgccggtcgggggcaccgggactgactctgcggccggcgcgggagggctgagggcaccttagaaacccagccccgagccaccccgaggagggagctgaggcacagagaggtagcacccctcctgaggtcacacagcgagtgagtggccaggatagaaacgaaggtctcggagcccagcactgtcccccatgcatcctcgccggctgggggcatagggaacacccagccgccgaggaagggggcagccgcggccaggggatggatgttcgatgccaggggaagccgggtgactgcagcagagaccctctcagcgcccctcgggggaggcttgtggccgatttggcccaatgatcgggtgcccaggttccctgcactctcaacatttgctccgtaaatttgtctttataaatgtcaggggtcggggagggctgcctccctacttaaaagcgccctgctcctctaggaaggcccgggcaggggccctgcgctgggcagggagctcgcttccctgttccgggctggccgggagccccagggtgtctcccaacaggtgggtccagcttctccctggggctcgttcatcctgggctgggtctgcccgacttgcgtgggtgggggatggtggcctgggctggcatgttgggggaacccccagcacctgctggcggcttggggcagtgagggggacgcagggtgatggggccactcgggcccctgggcggagtagcattataatggtgtattgtgtatttttcaatttcctaaaggtaactgttcagtcctcacctaattttacacagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcggtaaggcgcgctgaaggtagcttgtgggatgcgcggcgcctggaccaccagtcccaactgcactggccgggggcctgcagggtccccagctcagaagggaaggagttaaggcagaaaggtgtcttgagggtcagctggggaagagagggctagggaaggggcctcggggagtacttggccacaggtgcccccttccctgcccagcaaatgaatgaatctaggaatctccctttacacctccccccttcatacactcctccccagctgcaggtggaggcagggggttcagggggagggggcctaggtttaaggcaagcgcttgaaagaggatatgtcgccagggcccacaaaaggagagtgggaaagagaagggagagaaatcccctcaactctcccagttggaaaaggaggttgctgggaacccttaaatactttaatcggatgggtcaatttacacgtggaaactgggcttggaagggggctctccctgctccattccctggggaacccaggcggctggggcagggctcccggctccctgtggggtggcaagtgcggagagctactgccggcagagaggctgaggactgattgatttctaaaacattcaatattcctgccttgaaacggggccaatttccagagtcctggatgtaccaagagaaaagcaagccctctgggcggctggtgttgtttttagagagcgattattgtgagtttaaaaacctttaaataatgaccacgttaaacaagcatgaaaggagaggggaagactctattaaaatgtaataaaaaggtgaggcattttaagagctaagaagaaaaatgaagaatgatataaaacagaaaaagaacgagctttttttcagatgtggagtttgaaaggagttgcaagtctgtaaatgttaaaaaagaggttcaagattgttctctctgaatctctggagagacccctgcgtctggcagagcgattaaaactcttcttactttcaccttttttaacatttcctgaatactttctcctcttcttgagccagtttgggggatcgagtatgtctcgctgcattttttctgcatcgacgtagctgacttcccttcaaagctggaggagacataattttcctgggataaatctgtcactggggtggaagaataggtttgaaaatgttaagctttccaaaagcccggtcgggagagctgccgtaaactgcggccccagctccccagatcggagcagattcaaccgacatgggagagagcgggctttctggggtgggggtgggggcggcctgcctggtggccgagctcccctactgggctggcagagcaggcctaccatccctttgtccctccaaattagctgcttgctgcccttctcagcttctaaggagaggagacgaatgcctggctcttccagggtttgctctgtcagagcaaatggcccagcatccttggatttcggggagggaagcagaagtcaagctggtgtgggggaattcatcttcagacatcagaggaagcctgatgtccacttctctccgtggccatataagaggtttcccgccttccctagtccctgccccacagcaggataggagctggaggccctaagaacagaggctcagggtgttcagccaggacaggagtagtccggggagaatagcagagaggtctcaatggcttctttaccggccccaggaagatgcagaggcacctacttcatccctgcctgccgcatctggccagaatccccagagtggcatcctctcactcctccagtcagcatccttcctggcccttaaaaatagctgcctcttgctgtggaaggcagagcgggagagggatgagcagactggctaggtccaggaggaggtgggcaggcagggaggtgacagctgcatttccctgctgacttggggccaaaccgggggcttggccactggtcccagcactgagcctgtgtgaagcggggaacattacaagctgagcagtgatgactgacttgctgacctcgatgccccaaacccctgccatgggtttcagggacagaggaggtgccatatgtcaatgcaggctaagtccaagaactgctttctcagggaggggagaggctgggctctgcggcagggacggcaggagagccagcgccttctgtcccttcctaaacagtgaagtcaccctcactactttcttgaggacagcctataagagatgattgcacagagaggtgaacaggacaggcttctgtggcaagttggctgtctaaggggaggggaatgggaaaggaggcatagaattcagccctgcctcctggcctccagtctaagaggccagcaggcaggattctccctcctcctcacccacagaagctatttggtcactgggctgagaagcccagctgtgatgaggctgaaagagaaaagacaattgagttgttgatttctagacactgggctggggaccctggtgactgggaagccacagggaagaggcaggatcctcctggcaggtgtctgaggttctgggaaagcagcctgctttgggctgtgtgtttagggagagctgtccccatccctagcaccttctcagcagagcagagcctggtgatgttgaggggctgggggtggccaaagctgtgagttagaacccaaagtggcagaaacaggagacaatgctttcatctttggccttgggactggaggtcaccccttcacccaagtgtttgtaatgacctgtgagacacgcacacataatatttggagttggaaggatcctcggagatcttttgattagctatggaactctgattcaattaaattttacttagatcctcaatctaagagacatcactgtctggtttaaaaagagggtggaaggaatggcacttcagccttttcaatttgatagtcccaagacctctcttgagaacccagtttgaaaaccataatcccgtccactgccctctgtaaacccaggggtggggcccgaggtccagaaaagccctttctctgggttgcacagcatgttagtagctgggctggagctggagtccatgttttgggttcctggtctttggagcagttgctgctgggctgtgacaagtgtgtgggccagctggggtcagggatctgccaatagccatcctgctaccttctctgacagcaaagctcatcgtggagacggacacctttggaagcagagttcgagtccgaggagccgagacgggcctctacatctgcatgaacaagaaggggaagctgatcgccaaggtgaggccccagcaggcaggtggggtccgcgggacctagggagtggcttgggctggctggactcctccaagacagtcctgggctctgatcccaacaaggtaggcagaaggccacagggccatcttggggttacctcacgattgggtgcaggagtgagggagaaggaagctgtttgagagatgaaaactataaaaggcgaaggaggaagaatgaaggctagggaaggggctggggggtggtggttggtaaattccatatatctttttaacaaatcgccaaattgctttgctattatgtccaattacatggtaccagcatttggaatgttcagtaagccgcaggcccagcccctcggccgagctctgaaatggctccattagtcacggctcctctccagcctcgagctccccacttcctgggcttctggggctggggtcttagcatcttctcccaggcctcccctcccccatagggtggctgccctggggccagggaaccgaagtcctgggggggtgagaggggcaggtggggagacgggtggccagactggtgggcaggaggccagagcaggccaggctctgggcccctctctctgtctttctgcgttggggcccagcccctccgtagacaaccatgtgtcactgctgcctgggaggacaggaagttgccgggtgggctgcgagttgtgagggattagagagcgggtgcccaggcagggggtggggctgcggctcctgcccacctcgccatctgctggggtgcccacctgctgtctggggccgctcgccctctgcctctgctgggggggctctgtaacgtggtgtctggctcccctacctgcagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggcccccgagccccgataggtgctgcctggccctccccacaatgccagaccgcagagaggctcatcctgtagggcacccaaaactcaagcaagatgagctgtgcgctgctctgcaggctggggaggtgctgggggagccctgggttccggttgttgatattgtttgctgttgggtttttgctgtttttttttttttttttttttttaaaacaaaagagaggctctatttttgtattccacttggctgtggtgtctgtcttcttaactctcagaaagctccattagtggcctagactgggattccggctgggggtttgcgggggtggggggctttctctagcctgtgctgctgaggccccagtacctccagggccagttggctgggcagccagggactccactgcacccccaggtggggcagggaggaaaggactgtgacatagggcagtcctcttagaagtgggtatcagactggtggctattaaatgattgaaatatttatttaacttgcatattaaaaatgtgtgctggagagtgagtcctgccggggtcagcccctccctccaaccttgccccagctggtgggcggctgggagacgcagatgaccaggtgccagctctgaccacagcctccctccagcctaaagacacctgcctgtcaaccatccccatcactgtcacttgaggggttttcctgcaaggacagaagcagggaaaggggcaagaagaggctcttagctagtccttggagctctcagatgtgtacctcctagcactttacagaggtcattgctaacacttccccaggccacctcagggccagaaataatggatgtgctagggctagagctgtaatcatggatttaatcctcttaaaaagtgcttctctgagtgcctaggtccatgtgggagacaggttggagattccagaacttgctctttctgagactcaggctccagaaaatgaaagaaaagagcagctgccagggtccaaggtgggggcatattggagggggaccaccaagactggtgttgacaatggtgatgtgggacaagtgttaaccttgggtgatatggtgagatagctgtgggcagaaagcactgagctgaggtgcggtgaggagcctggggaactgtcttccaggaagaggctgcccacctcggaggatgggcctggcgggagaggagctgggcaccggatggcaccagaagggaagctcataggcctagcgcagaactaaaggcagtcatagccttggggagaagcaggaggccgtatgtggagggagggagggctgctgtgggagtggtggagcaggtcatggtgtgggcagagaagggaatgggcaagggtgcaggtgtgtgtttgcgtgtggactggtgagactggtgtcctgccacaccgagggagagcccaggccccacggcagtttcctgagtgcagagctggcccaggcttcatcgctgaggcctcccattagggctgctcctgcttccttccttgtggatgccctgggctggtcccacagcccagctactgagccagtctagaaacctcgctcctctaggactctgtccattggcatctcctggggtagggagcaacccaaggcttcctcccacagtttcaggcccaagtcccaggtttagcccaccccggcagatcaggaatttcgaggcttagtgggaagcccggcagtaaaggctggcctgtccagccacctctcgtggcctgccacaggcggtgaggggctctggagagagtgggtttctgctggtgcgtagacactggatgtagcgaaggggccctagatgtctctcttccagttcccccagctcttagcgcctcctcctctgctcccttccccctttaattaattctgggtaagcagcctggctttattgtgcaaggagctggggtctcactgtgggaaaagtcacaccttctgagcagcttctgatgccatgcaaatgaagggaccgtccaggaaacgctttcatctgcacggtctcctgtccagcccctagcccagtaattacccacatttcatttgtttgagagcaattcctg

SEQ ID NO: 4 - protein, 244 aa, human
MGSPRSALSCLLLHLLVLCLQAQEGPGRGPALGRELASLFRAGREPQGVSQQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 5 - DNA, 1238 bp, human
  CDS (13)..(663)
acctctccagcgatgggagccgcccgcctgctgcccaacctcactctgtgcttacagctgctgattctctgctgtcaaactcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgtagtctgggaggcagggggcagcagcccctgggccgcctccccacccctttcccttcttaatccaaggactgggctggggtggcgggaggggagccagatccccgagggaggaccctgagggccgcgaagcatccgagcccccagctgggaaggggcaggccggtgccccaggggcggctggcacagtgcccccttcccggacgggtggcaggccctggagaggaactgagtgtcaccctgatctcaggccaccagcctctgccggcctcccagccgggctcctgaagcccgctgaaaggtcagcgactgaaggccttgcagacaaccgtctggaggtggctgtcctcaaaatctgcttctcggatctccctcagtctgcccccagcccccaaactcctcctggctagactgtaggaagggacttttgtttgtttgtttgtttcaggaaaaaagaaagggagagagaggaaaatagagggttgtccactcctcacattccacgacccaggcctgcaccccacccccaactcccagccccggaataaaaccattttcctgcaaaaaaaaaaaaaaaaaa

SEQ ID NO: 6 - protein, 216 aa, human
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT